(19) United States
(12) Patent Application Publication
(10) Pub. No.: US 2023/0094567 A1
(43) Pub. Date: Mar. 30, 2023

(54) LASER FIBER INTEGRATED MORCELLATOR

(71) Applicant: GYRUS ACMI, INC D/B/A OLYMPUS SURGICAL TECHNOLOGIES AMERICA, Westborough, MA (US)

(72) Inventors: Nikhil M. Murdeshwar, Maple Grove, MN (US); Peter J. Crowley, Norfolk, MA (US)

(73) Assignee: GYRUS ACMI, INC D/B/A OLYMPUS SURGICAL TECHNOLOGIES AMERICA, Westborough, MA (US)

(21) Appl. No.: 17/935,489

(22) Filed: Sep. 26, 2022

Related U.S. Application Data
(60) Provisional application No. 63/261,716, filed on Sep. 27, 2021.

(51) Int. Cl. A61B 18/14

(52) U.S. Cl. CPC A61B 18/14; A61B 2018/00547

(57) ABSTRACT

Various examples disclosed relate to treatment of BPH with an integrated device for both tissue treatment and removal. The present disclosure includes a device including a sheath for partial insertion into a patient, a laser fiber actuatable for laser enucleation of tissue, an elongated member, and a tissue engagement tool on the elongated member.

### PRIORITY CLAIM

This application claims the benefit of priority to U.S. Provisional Patent Application Ser. No. 63/261,716, filed Sep. 27, 2021, the contents of which are incorporated herein by reference in their entirety.

### BACKGROUND

Benign Prostatic Hyperplasia (BPH) increasingly affects men as they age. BPH can cause enlarged prostate tissue. This enlarged prostate can cause a number of uncomfortable urinary conditions, including significantly restricting urine flow, urethra stricture, frequent urination, inability to urinate, difficulty in starting urination, or loss of bladder control, and other complications.

### SUMMARY OF THE DISCLOSURE

The present disclosure provides devices and methods for treating BPH. The devices can include a multi-use tool for urology. The tool integrates a laser fiber with a morcellator. This can allow for enucleation and morcellation to be combined in a single step procedure. This can shorten procedure time and improve safety.

A variety of techniques are used to medically address BPH. These can include observation, dietary, medicinal, and minimally invasive surgical techniques. Medicinal approaches can include alpha blockers, 5-ARI or PDE-51, while minimally invasive techniques can include TUMT, TUNA, steam vapor, and others. In some cases, invasive surgeries are used, such as TURP, TUIP, HoLAP, green light, HoLEP, or prostatectomy. Certain techniques are useable for prostates of particular sizes. Some of these surgical techniques can used plasma energy, bipolar energy, or laser energy, to trim, cut, or reduce enlarged prostate tissue.

In some cases, prostate tissue is removed through enucleation or resection methods, where the trimmed or cut away tissue is directed into the bladder. In this case, a separate tool, such as a morcellator, can then be used to remove the tissue from the bladder.

However, these types of surgical techniques rely on two distinct steps, with multiple tools, for full removal of prostate tissue. The discrete steps include first severing targeted prostate tissue (e.g., with plasma energy, bipolar energy, or laser energy), moving that resected tissue to the bladder, and then using a separate device (e.g., a morcellator) to further cut and retrieve the tissue from the bladder.

Such multi-step and multi-instrument techniques take a significant amount of time to perform. Additionally, complications can occur at multiple steps. For example, anatomy can be damaged, such as causing urethral sphincter. This can occur where the field of vision is blocked by the resected prostate tissue itself, obscuring a view of anatomical landmarks.

A reduction in surgery times, hospital stays, and post-procedure catheterization would be beneficial. Additionally, methods that allow for better safety by maintain the field of vision are desired. The single tool, combined methodology discussed herein can allow for contemporaneous resection and cleaning of prostate tissue.

In an example, a device for at least partial insertion into a patient can include an elongated sheath having a proximal portion, a distal portion and a lumen, the sheath for partial insertion into a patient, a laser fiber extending longitudinally within the lumen of the elongated sheath, the laser fiber actuatable for laser enucleation of tissue at the distal portion of the elongated sheath, an elongated member extending longitudinally within the lumen of the elongated sheath, the elongated member extending concurrently with the laser fiber and a tissue engagement tool at a distal portion of the elongated member, the tissue engagement tool actuatable for removal of tissue. The laser fiber and the elongated member can be configured for concurrent insertion into or removal from the lumen of the sheath.

In an example, a method of treating prostate tissue can include enucleating the prostate tissue with a laser fiber and morcellating the prostate tissue with a tissue removal device, wherein the laser fiber and the tissue removal device are integrated into a single medical instrument.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIGS. 1A-1C illustrate an example multi-use tool for resection and clearing of prostate tissue.

FIGS. 2A-2C illustrate an example multi-use tool for resection and clearing of prostate tissue.

FIG. 3 illustrates an example multi-use tool for resection and clearing of prostate tissue.

FIGS. 4A-4C illustrate an example multi-use tool for resection and clearing of prostate tissue.

FIGS. 5A-5D illustrate an example device for medication of treated prostate tissue.

FIGS. 6A-6B illustrate an example device for medication of treated prostate tissue.

FIG. 7 illustrates an example method for resection and clearing of prostate tissue.

### DETAILED DESCRIPTION

The present disclosure describes, among other things, a single device or tool for surgical treatment of BPH by resection and removal of prostate tissue in a single surgical step. In an example, the device can include a laser fiber and a morcellator. This can allow for the two steps of enucleation and morcellation to be combined into a single step procedure, shortening procedure time. This can additionally improve safety and reduce the chance of damage to the bladder during the procedure.

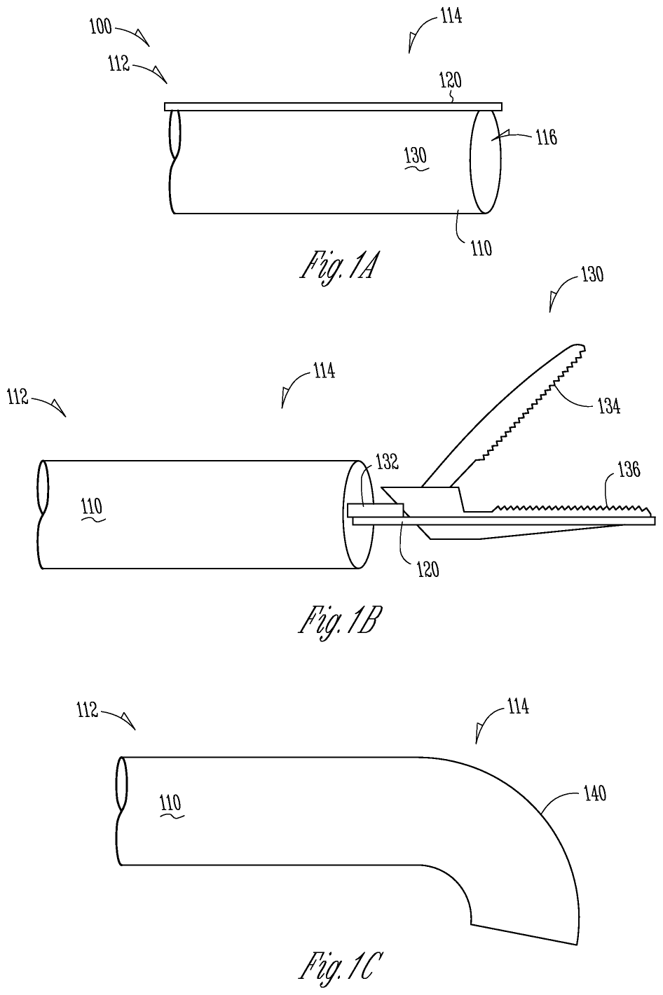

FIGS. 1A-1C illustrate an example multi-use device 100 for resection and clearing of prostate tissue in different operations. The device 100 can include an elongated sheath 110 having a proximal portion 112, a distal portion 114, and a lumen 116, a laser fiber 120, and a tissue engagement tool 130 extending from an elongated member 132. Shown in FIG. 1C, the device 100 can further include a distal segment 140.

In device 100, the lumen 116 of the elongated sheath 110 can host both the laser fiber 120 and the elongated member 132. The laser fiber 120 and the elongated member 132 can extend longitudinally at least partially within the lumen 116 of the sheath 110, such that the laser fiber 120 can extend out the distal portion 114 of the sheath 110 to allow for laser enucleation of tissue. Similarly, the elongated member 132 can extend out the distal portion 114 of the sheath 110 and terminate with the tissue engagement tool 130 to allow for grasping of tissue at the distal portion 114.

The elongated sheath 110, extending between the proximal portion 112 and the a distal portion 114, can be a sheath or shaft shaped and sized for at least partial insertion into a patient, such as laparoscopically or through the urethra, for treatment of BPH by enucleation and removal of excess prostate tissue. The sheath 110 can include a central longitudinal lumen 116, through which both the laser fiber 120 and the tissue engagement tool shaft 132 can extend. During operation, a surgeon can grasp or hold the sheath 110 near the proximal portion 112, such a at a handpiece or handle (not shown) and manipulate the device 100 into the patient. For example, the surgeon can manipulate the device 100 up through the surgical pathway towards the prostate to enucleation and remove enlarged prostate tissue.

The laser fiber 120 can run through the lumen 116 of the sheath 110, such as from a laser source (not shown) attached to the proximal portion 112, towards the opening of the lumen 116 at the distal portion 114. In FIG. 1A, the laser fiber 120 is shown along the body of the sheath 110. In some cases, a groove can be provided in the sheath 110 along the side of the lumen 116 for placement of the laser fiber 120. In contrast, the example shown in FIG. 1B, the laser fiber 120 is situated adjacent the tissue engagement tool 130 elongated member 132 extending down the center of the sheath 110 lumen 116.

In the example of FIGS. 1A-1C, a laser fiber is shown for treatment of enlarged prostate tissue. Various endoscopic surgical methods can be used in treatment of BPH, and variants on the laser fiber 120 can be substituted. The laser fiber 120, depending on its configuration, can be actuatable for enucleation, resection, or vaporization of prostate tissue. In enucleation methods, the laser fiber 120 can be an end-firing laser used to peel the entire prostate adenoma from the prostate capsule. In some vaporization cases, the laser fiber 120 can eb replaced with a designed bipolar loop. In resection, the prostate tissue can be moved piece by piece, and the laser fiber 120 can be replaced by a mono-polar or bi-polar resection loop. In vaporization, the laser fiber 120 can be a side firing laser fiber to vaporize the enlarged prostate adenoma, or alternatively the laser fiber 120 can be replaced with a mushroom-like bipolar electrode.

In an example, the laser fiber 120 can be a laser fiber configured for holmium laser enucleation of the prostate (HoLEP), or alternatively a laser configured for laser vaporization of the prostate. In the case of HoLEP, the laser is used to cut and remove the bulky prostate tissue that is blocking the flow of urine. Specifically, HoLEP can include cutting a short surgical horizontal incision to exposed the surgical capsule of the prostate before the apex. The surgeon can dig a tunnel from the apex to the bladder neck beneath the median lobe along the surgical capsule. The surgeon can then enucleate the prostate adenoma in a counterclockwise manner in the first lobe, then clockwise in the second lobe. The surgeon can then push the entire prostate off the prostatic wall and into the bladder.

Once prostate tissue is removed, whether by enucleation, resection, vaporization, or other methods using the laser fiber 120, the tissue engagement tool 130 can be used to remove the tissue. In conventional methods, this would require a separate tool, such as an independent morcellator. Morcellation of tissue can include “mincing” of tissue to smaller pieces of tissue, or remnants, for removal from the body.

However, the device 100 is an integrated device with both the laser fiber 120 to treat prostate tissue and the tissue engagement tool 130 to remove the treated tissue. The device 100 can combine both tissue treatment (e.g., enucleation or vaporization) and tissue removal (e.g., morcellation) steps into a single-step procedure, shortening the procedure time. This can help avoid bladder damage and improve safety by keeping the field of view open.

The tissue engagement tool 130 can extend distally from the elongated member 132. In some cases, the tissue engagement tool 130 is a morcellator configured to collect, digest, and remove prostate tissue that has been treated with the laser fiber 120.

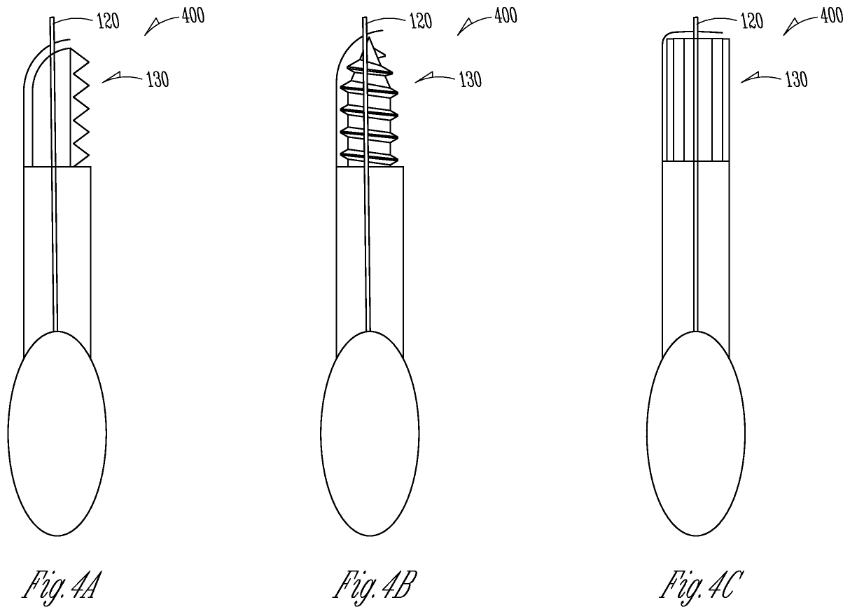

In some cases, the tissue engagement tool 130 can be a morcellator. In this case, the morcellator tissue engagement tool 130 can include a shaft having a blade (see FIGS. 4A-4C) that is configured to further chew up tissue. In this case, the laser 120 can be actuated to enucleation prostate tissue, which is then sucked into the shaft of the morcellator tissue engagement tool 130 and cut up before disposal. Such a morcellator can be a bipolar, a monopolar, or a mechanical morcellator.

Where the tissue engagement tool 130 is a morcellator, the distal segment 140 can be included. The distal segment 140 can, for example, be a flexible segment covering the tissue engagement tool 130. The distal segment 140 can be configured to deflect against tissue to improve access to the tissue.

In some cases, the tissue engagement tool 130 can instead be a forceps jaw for mechanically grasping the prostate tissue. In this case, the forceps tissue engagement tool 130 can be moved and clamped around tissue to be removed. The forceps tissue engagement tool 130 can hold the tissue in place, while the laser 120 is actuated to vaporize or enucleate the tissue. The forceps tissue engagement tool 130 can then be moved to another portion of tissue. As needed, the forceps tissue engagement tool 130 can be used to pull remainder tissue out.

In the case of a forceps tissue engagement tool 130, the laser fiber 120 can be integrated into the tissue engagement tool 130, such that the forceps tissue engagement tool 130 and the laser fiber 120 work in conjunction for enucleation and vaporization of tissue.

A variety of examples of the integrated device 100 are shown in FIGS. 1A-1C.

For instance, FIG. 1A depicts an example where the laser fiber 120 is positioned along the sheath 110, which may be positioned in a groove to protect the fiber. In this case, the tissue engagement tool 130 can be a morcellator integrated into the sheath 110, such that the laser fiber 120 sits within the morcellator itself. In FIG. 1A, the laser fiber 120 can be used to cut prostate tissue, with the morcellator 130 further severing tissue if needed, and removing the prostate tissue.

By comparison, FIG. 1B depicts an example where the laser fiber 120 is positioned on the back of the forceps tissue engagement tool 130. In this manner, the laser fiber 120 can be used to cut/resect tissue, and forceps tissue engagement tool 130 can remove tissue pieces as needed. FIG. 1C depicts a flexible morcellator tip 140 used in combination with forceps tissue engagement tool 130, thereby improving access, and improving visibility during the procedure and allowing tracking of anatomical landmarks.

In some cases, the tissue engagement tool 130 can be a morcellator. In an example, it can be a bipolar morcellator. Such a bipolar morcellator can include a jaw for grasping tissue and at least two electrodes. The two electrode can provide radio frequency (RF) energy for morcellation. In some cases, the morcellator can be a monopolar morcellator. In some cases, the tissue engagement tool 130 can be a forceps jaw with a vaporization feature. In some cases, the morcellator can be a mechanical morcellator, such as a blade, screw, or teeth.

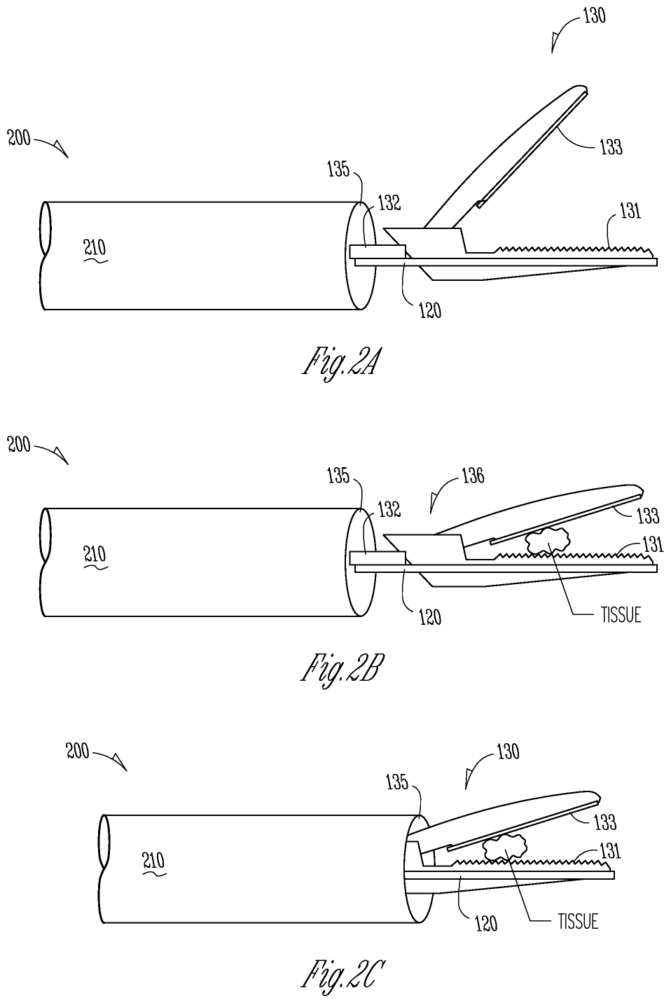

FIGS. 2A-2C illustrate an example multi-use device 200 for resection and clearing of prostate tissue. The device 200 is similar to device 100, and contains similar components. The device 200 specifically depicts an example forceps tissue engagement tool 130 working with a laser fiber 120 to enucleate and vaporize tissue. The device 200 uses a bipolar morcellator for the tissue engagement tool 130, compared to the examples of device 100 above.

In FIG. 2A, the laser fiber 120 can be used to resect or enucleate tissue. Then, in FIG. 2B, the forceps tissue engagement tool 130 can secure pieces of tissue. In FIG. 2C, the forceps tissue engagement tool 130 can be retracted with the pieces of tissue to electrically morcellate the tissue.

The multi-use device 200 can include a bipolar arrangement for the forceps tissue engagement tool 130, where a first electrode 133 is on a jaw 131 of the forceps tissue engagement tool 130. A second electrode 135 can be on the sheath 110. The first and second electrodes 133, 135, can be of opposing polarities. Thus, when the forceps tissue engagement tool 130 is retracted through the sheath 110 (FIG. 2C), the forceps tissue engagement tool 130 first electrode 133 and the second electrode 135 can create an electrical path. The electrical path can be sufficient to electrically morcellate the secured tissue pieces. The remnants of the morcellated tissue can be drawn out through the sheath 110 or by further retraction of the forceps tissue engagement tool 130. In some cases, the remnants of the morcellated tissue can be pushed into a holding area in the device 200. In some cases, suction can be used to draw the remnants out of the body.

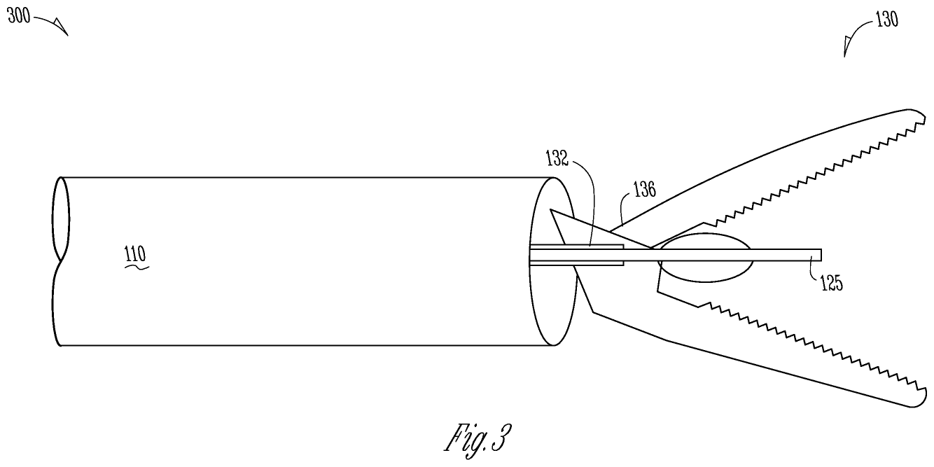

FIG. 3 illustrates an example multi-use device 300 for resection and clearing of prostate tissue. The device 300 is similar to device 100, and contains similar components, except where otherwise noted. The device 300 specifically depicts an example forceps tissue engagement tool 130 working with a laser fiber 120 to enucleate and vaporize tissue. The device 300 uses a vaporizing laser fiber 125.

In device 300, instead of using bipolar energy to morcellate the tissue, a separate laser fiber 125 can be used to vaporize grasped tissue. The laser fiber 125 can be positioned at the pivot point 136 of the forceps jaw 131, and extend distally between the jaws. When tissue is grasped between the forceps jaws 131, the laser fiber 125 can be used to vaporize the tissue. Thus, device 300 does not include a separate morcellator.

FIGS. 4A-4C illustrate an example multi-use device 400 for resection and clearing of prostate tissue. FIGS. 4A-4C depict examples of the tissue engagement tool 130 that include mechanical morcellators. Such a mechanical morcellator tissue engagement tool 130 can reside partially or fully within the sheath 110. The mechanical morcellator tissue engagement tool 130 can be configured to receive enucleated tissue, such as that treated with the laser fiber 120, and further morcellate the tissue.

In FIG. 4A, the mechanical morcellator tissue engagement tool 130 can include a blade with a number of teeth. In FIG. 4B, the mechanical morcellator tissue engagement tool 130 can include a screw-like blade. In FIG. 4C, the mechanical morcellator tissue engagement tool 130 can include plurality of blades. These mechanical morcellator tissue engagement tools 130 can be configured to rotate, so as to further mince or chew up received tissue.

Each of the devices shown in FIGS. 4A-4C can involve a laser fiber being provided through a central lumen of the mechanical morcellator. The laser fiber may alternatively be provided elsewhere such as along an edge of the morcellator, or in any other non-central location capable of receiving the laser fiber therethrough.

Each of the devices 100, 200, 300, 400, can combine the two steps of tissue treatment (e.g., enucleation or vaporization) with tissue removal (e.g., morcellation), into a one-step procedure, with optional continuous removal of tissue during the operation. This can aid in shortening procedure times.

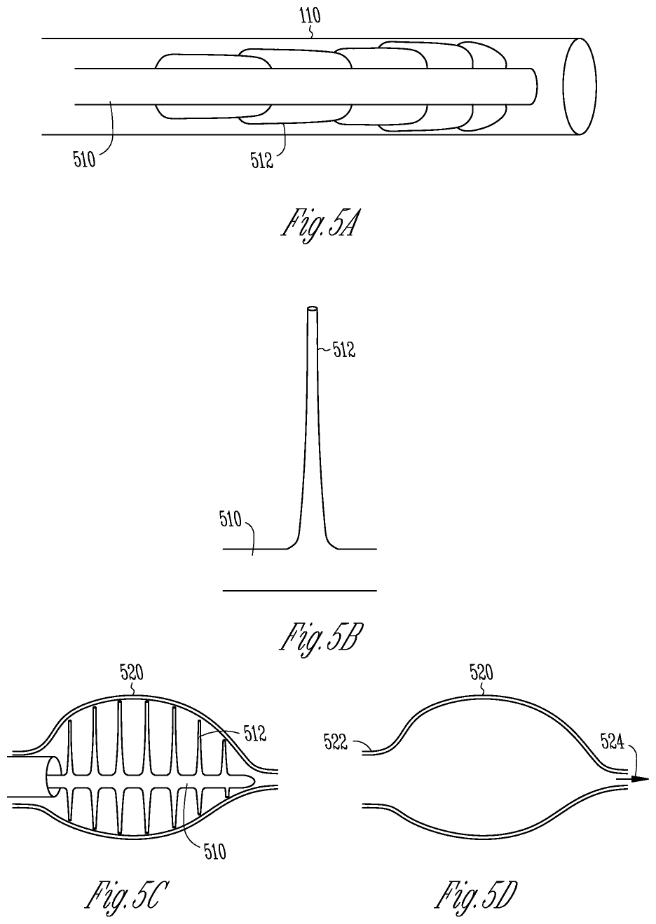

FIGS. 5A-5D illustrate an example device 500 for medication of treated prostate tissue. Another example involves controlling strictures and facilitating healing after the procedure, as depicted in FIGS. 5A-5D. One example involves using a medicated gel. For example, a bristled device may be delivered such that its bristles collapse during delivery, and expand to a gel delivery shape upon exiting the delivery device.

The device 500 can include the sheath 110 extending between the proximal portion 112 and the distal portion 114 with the lumen 116. The device 500 can further include a tube 510 with bristles 512 for carrying a medicated gel. The device 500 can be inserted into the prostate cavity 520 along the urethra 522 towards the bladder 524.

In FIG. 5A-5D, the sheath 110 can include the lumen 116, and the medication delivery tube 510 can be used to deliver medicated gel along the length of the lumen 116 to the prostate cavity 520. The tube 510 can be sized and shaped for insertion down the lumen 116 towards the prostate tissue and cavity 520. The tube 510 can optionally include bristles 512, which can flex along the length of the lumen 116 during delivery.

The bristles 512 can be on the tube 510 and straighten when they are pushed into the prostate cavity 520. The bristles can serve several functions, including serving as a conduit for the mediated gel to move from the tube into the cavity, and as a brush to spread the gel on the cavity. The bristles 512 can, for example, include lumens or hollow portions to allow spreading of gel therethrough, When the bristles 512 reach the cavity 520, the bristles 512 can vertically expand, and the medicated gel can be dispersed through the bristles. The tube 510 can be moved forward and back and rotated, helping to spread the medicated gel along the walls of the prostate cavity 520.

Specifically, the prostate cavity 520 can be treated by a medicated gel with device 500 following laser enucleation or vaporization. In this case, the tube 510 can be inserted down the lumen 116 of the sheath 110 either contemporaneously with the laser fiber 120 and/or the tissue capture device 130, or after the laser fiber 120 and/or the tissue capture device are retracted along the sheath 110. In some cases, the tube 510 can be inserted separately up the endoscope after the multi-use device is used for treatment.

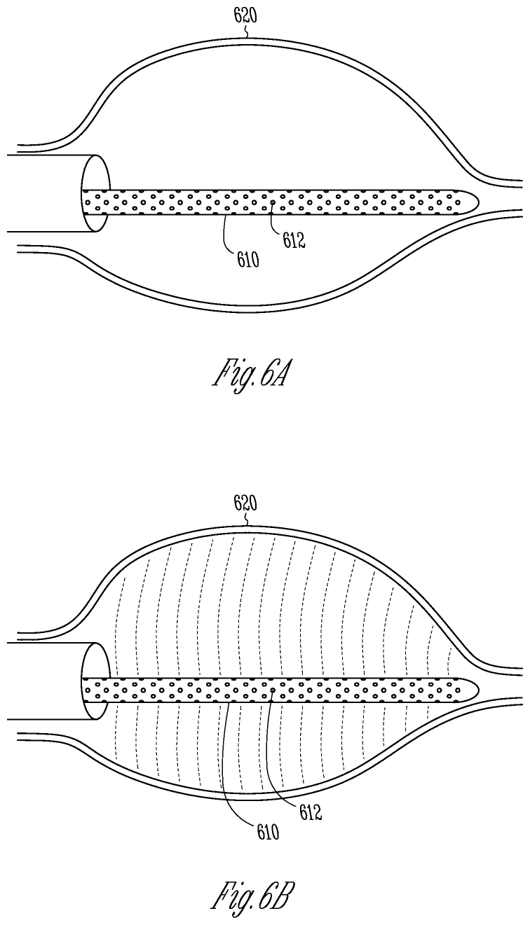

FIGS. 6A-6B illustrate an example device 600 for medication of treated prostate tissue. The device 600 can be used, for example, to provide a medicated spray to the prostate cavity 630.

The device 600 can include a tube 610 with a plurality of spray holes 612. In this case, the tube 610 can be inserted along the sheath 110 lumen 116 or other endoscope after use of the multi-use device for treatment and removal of prostate tissue. Once inserted into the prostate cavity 630, a medicated spray can be released along the length of the tube 610 and through the spray holes 612 to spray along the interior surface of the prostate cavity 630. In some cases, the spray can be originally in a liquid state for delivery, and can be configured to change to a gelatinous state when the temperature of the spray changes as a result of being applied to the prostate.

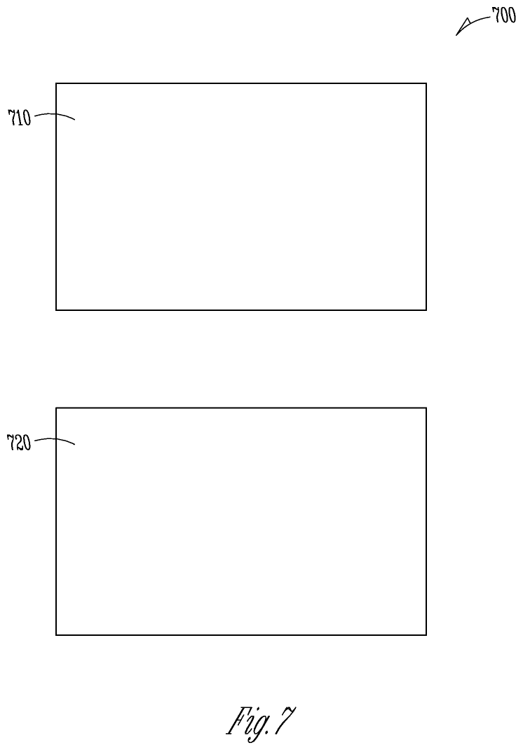

FIG. 7 illustrates an example method 700 for resection and clearing of prostate tissue. The method 700 can include blocks 710 and 720. At block 710, the prostate tissue can be enucleated with a laser fiber. At block 720, the prostate tissue can be morcellated with a tissue removal device. The laser fiber and the tissue removal device can be integrated into a single medical instrument. Morcellating the prostate tissue can include application of radio frequency energy with a bipolar morcellator. Morcellating the prostate tissue can include mechanical morcellation of the tissue.

### Various Notes & Examples

Example 1 can include a device for at least partial insertion into a patient, the device comprising: an elongated sheath having a proximal portion, a distal portion and a lumen, the sheath for partial insertion into a patient; a laser fiber extending longitudinally with the elongated sheath, the laser fiber actuatable for laser enucleation of tissue at the distal portion of the elongated sheath; an elongated member extending longitudinally within the lumen of the elongated sheath; and a tissue engagement tool at a distal portion of the elongated member, the tissue engagement tool actuatable for morcellation of tissue.

Example 2 can include Example 1, wherein the laser fiber and the elongated member are configured to move together relative to the sheath.

Example 3 can include Example 1, wherein the laser fiber is located on an outer surface of the elongated sheath.

Example 4 can include Example 3, wherein the outer surface of the sheath comprises at least one groove for engaging with the laser fiber.

Example 5 can include Example 1, wherein the tissue engagement tool is a morcellator.

Example 6 can include Example 5, wherein the morcellator comprises a bipolar morcellator comprising jaws actuatable for grasping tissue and at least two electrodes actuatable for providing radio frequency energy to the tissue.

Example 7 can include Example 6, wherein the laser fiber extends between the jaws.

Example 8 can include Example 5, wherein the morcellator comprises a mechanical morcellator.

Example 9 can include Example 8, wherein the morcellator comprises one or more blades with teeth, a rotatable screw, or a plurality of rotatable blades.

Example 10 can include Example 1, wherein the tissue engagement tool comprises a mechanical jaw.

Example 11 can include Example 10, wherein the mechanical jaw is actuatable for blunt dissection of tissue.

Example 12 can include Example 10, wherein the mechanical jaw and the laser fiber are actuatable for vaporizing tissue.

Example 13 can include Example 1, further comprising a deflected segment on the distal portion of the elongated sheath.

Example 14 can include Example 1, further comprising a delivery tube configured to delivery of a medicated gel, the delivery tube situated within the lumen of the elongated sheath.

Example 15 can include Example 14, wherein the delivery tube further comprising a plurality of bristles extending laterally outward actuatable for dispersing the medicated gel.

Example 16 can include Example 1, further comprising a delivery tube configured to delivery of a medicated spray, the delivery tube situated within the lumen of the elongated sheath.

Example 17 can include Example 16, wherein the delivery tube further comprises a plurality of spray holes actuatable for dispensing the medicated spray.

Example 18 can include a method of treating prostate tissue, comprising: enucleating the prostate tissue with a laser fiber; and morcellating the prostate tissue with a tissue removal device, wherein the laser fiber and the tissue removal device are integrated into a single medical instrument.

Example 19 can include Example 18, wherein morcellating the prostate tissue comprises application of radio frequency energy with a bipolar morcellator.

Example 20 can include Example 18, wherein morcellating the prostate tissue comprises mechanical morcellation of the prostate tissue.

Each of these non-limiting examples can stand on its own, or can be combined in various permutations or combinations with one or more of the other examples.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

What is claimed is:

1. A device for at least partial insertion into a patient, the device comprising:
an elongated sheath having a proximal portion, a distal portion and a lumen, the sheath for partial insertion into a patient;
a laser fiber extending longitudinally with the elongated sheath, the laser fiber actuatable for laser enucleation of tissue at the distal portion of the elongated sheath;
an elongated member extending longitudinally within the lumen of the elongated sheath; and
a tissue engagement tool at a distal portion of the elongated member, the tissue engagement tool actuatable for morcellation of tissue.

2. The device of claim 1, wherein the laser fiber and the elongated member are configured to move together relative to the sheath.

3. The device of claim 1, wherein the laser fiber is located on an outer surface of the elongated sheath.

4. The device of claim 3, wherein the outer surface of the sheath comprises at least one groove for engaging with the laser fiber.

5. The device of claim 1, wherein the tissue engagement tool is a morcellator.

6. The device of claim 5, wherein the morcellator comprises a bipolar morcellator comprising jaws actuatable for grasping tissue and at least two electrodes actuatable for providing radio frequency energy to the tissue.

7. The device of claim 6, wherein the laser fiber extends between the jaws.

8. The device of claim 5, wherein the morcellator comprises a mechanical morcellator.

9. The device of claim 8, wherein the morcellator comprises one or more blades with teeth, a rotatable screw, or a plurality of rotatable blades.

10. The device of claim 1, wherein the tissue engagement tool comprises a mechanical jaw.

11. The device of claim 10, wherein the mechanical jaw is actuatable for blunt dissection of tissue.

12. The device of claim 10, wherein the mechanical jaw and the laser fiber are actuatable for vaporizing tissue.

13. The device of claim 1, further comprising a deflected segment on the distal portion of the elongated sheath.

14. The device of claim 1, further comprising a delivery tube configured to delivery of a medicated gel, the delivery tube situated within the lumen of the elongated sheath.

15. The device of claim 14, wherein the delivery tube further comprising a plurality of bristles extending laterally outward actuatable for dispersing the medicated gel.

16. The device of claim 1, further comprising a delivery tube configured to delivery of a medicated spray, the delivery tube situated within the lumen of the elongated sheath.

17. The device of claim 16, wherein the delivery tube further comprises a plurality of spray holes actuatable for dispensing the medicated spray.

18. A method of treating prostate tissue, comprising:
enucleating the prostate tissue with a laser fiber; and
morcellating the prostate tissue with a tissue removal device, wherein the laser fiber and the tissue removal device are integrated into a single medical instrument.

19. The method of claim 18, wherein morcellating the prostate tissue comprises application of radio frequency energy with a bipolar morcellator.

20. The method of claim 18, wherein morcellating the prostate tissue comprises mechanical morcellation of the prostate tissue.
